(19) United States
(12) Patent Application Publication
(10) Pub. No.: US 2011/0087258 A1
(43) Pub. Date: Apr. 14, 2011

(54) CANNULATED ARTHROSCOPIC KNIFE

(76) Inventor: Robert K. Sluss, Naples, FL (US)

(21) Appl. No.: 12/888,039

(22) Filed: Sep. 22, 2010

Related U.S. Application Data
(60) Provisional application No. 61/251,614, filed on Oct. 14, 2009.

(51) Int. Cl. A61B 17/32; A61B 19/00

(52) U.S. Cl. 606/170; 128/898

(57) ABSTRACT

A surgical system for penetrating and cutting tissue. The system comprises a tissue penetrating/cutting assembly comprising a cannulated dilation sleeve which receives a movable knife. The knife barrel is configured to be received in the sleeve. The sleeve is also configured to receive a guide wire disposed in parallel with the knife barrel. The sleeve has a handle provided with a spring or friction slide to advance or retract the sleeve relative to the knife barrel to expose or retract the knife blade at the distal end of the sleeve .

This application claims the benefit of U.S. Provisional Application No. 61/251,614, filed Oct, 14, 2009, the entire disclosure of which is incorporated by reference herein.

### FIELD OF THE INVENTION

The present invention relates to surgical devices and, more particularly, to a cannulated dilatation sleeve combined with a knife.

### BACKGROUND OF THE INVENTION

During surgery (such as hip arthroscopy, for example), portals or small incisions are made on the skin to pass small instruments into the joint. These portals or small incisions are typically made in a “stepwise” fashion. The surgeon will insert first a spinal needle into the joint, and then a flexible wire into the joint through the spinal needle. The flexible wire serves as a place marker, but also allows for easy passage of cannulated instruments over the wire and into the joint.

An important step during hip arthroscopy is introducing a long handled scalpel (or knife) into the joint through a portal, to cut the hip capsule. The thick hip capsule normally restrains the movement of instruments and, when a small window is cut into the thick capsule, the cut allows significantly more freedom of movement of instruments in the constrained hip joint.

Once a small window has been cut, the surgeons introduce a long blade or knife through a cannula into the joint, to protect the surrounding soft tissue and neurovascular structures, or they simply put the blade through the skin portal directly into the joint and take a chance.

Accordingly, there is a need for a combined cannulated dilation sleeve with a knife (scalpel) that is integral with the cannulated sleeve. Also needed is an improved tissue penetration device that minimizes the risk of damaging adjacent organs and tissue during the cutting or puncturing step. There is also a need for a simplified surgical procedure to form a cut or puncture within tissue.

### SUMMARY OF THE INVENTION

The present invention provides surgical systems and methods for penetrating tissue. The system of the present invention comprises a tissue penetrating assembly comprising a cannulated dilation sleeve combined with (integrated with) a knife. In an exemplary embodiment, the knife is configured to retract up into the body of the sleeve. The sleeve is a double lumen tube to allow the knife to retract within one of the lumens of the tube. The sleeve is mounted on a handle provided with a spring or friction slide to advance and retract the knife blade barrel. The integrated cannula/knife is configured to be placed over a guide wire.

The combined cannula/knife system of the present invention saves significant time during surgery, as the cannula does not require a separate step for being introduced before the knife, since the cannula and the knife are combined. The combined cannula/knife system of the present invention also protects soft tissue and organs adjacent the cut, as the knife is retracted up into the protective sleeve once it has been introduced into the patient.

The present invention also provides a method of penetrating tissue by inter alia: (i) providing a combined cannulated dilation sleeve integrated with a knife, in the proximity of tissue to be cut or punctured; and (ii) deploying the knife from a lumen of the sleeve to cut or puncture tissue.

In an exemplary and illustrative embodiment only, a method of portal placement and capsular resection during hip arthroscopy according to the present invention comprises the steps of: (i) introducing a spinal needle; (ii) introducing a flexible guide wire through the needle; (iii) removing the needle, leaving the guide wire in place; (iv) sliding a cannulated retractable arthroscopic knife over the guide wire and into the joint, with the knife blade retracted back into sleeve; (v) removing the guide wire; (vi) deploying the knife to cut the capsule; (vii) retracting the knife blade into the housing, and reintroducing the guide wire through the knife cannulation; and (viii) removing the retractable knife from the joint, leaving the guide wire in place.

These and other features and advantages of the invention will be more apparent from the following detailed description that is provided in connection with the accompanying drawings and illustrated exemplary embodiments of the invention.

### BRIEF DESCRIPTION OF THE DRAWINGS

FIG. 1 illustrates a combined cannulated sleeve/knife according to an exemplary embodiment of the present invention.

FIG. 2 illustrates an enlarged view of the distal end of the knife assembly received in the combined cannulated sleeve/knife of FIG. 1, without the knife blade installed.

FIG. 3 illustrates an enlarged view of the distal end of the knife assembly received in the combined cannulated sleeve/knife of FIG. 1, with the knife blade installed.

FIG. 4 illustrates an enlarged view of the distal end of the combined cannulated sleeve/knife of FIG. 1.

FIG. 5 illustrates an enlarged end view (taken from the most distal end) of the combined cannulated sleeve/knife of FIG. 1.

FIG. 6 illustrates a side view of the combined cannulated sleeve/knife of FIG. 1 and placed over a guide wire.

FIG. 7 illustrates a front end view of the tubular guide or sleeve in an embodiment of the invention with separate cannulas for the knife assembly and guide wire.

FIG. 8 illustrates a side cross-sectional view of the tubular guide or sleeve of FIG. 7.

FIG. 9 illustrates a perspective view of the tubular guide or sleeve of FIG. 7 with a guide wire inserted therethrough.

FIG. 10 illustrates a perspective view of the combined cannulated sleeve/knife embodiment of FIG. 7 with a guide wire and knife assembly extending through respective cannulas and with the knife in a deployed or operative position.

### DETAILED DESCRIPTION OF THE PREFERRED EMBODIMENTS

The present invention provides systems and methods for penetrating tissue. The system of the present invention comprises a tissue penetrating assembly comprising a cannulated dilation sleeve integrated with a knife. In an exemplary embodiment, the knife is configured to retract up into the body of the sleeve. The sleeve is a double lumen tube to allow the knife to retract within one of the lumens of the tube. The sleeve is mounted on a handle provided with a spring or friction slide to advance and retract the knife blade barrel. The integrated cannula/knife is configured to be placed over a guide wire.

The combined cannula/knife system of the present invention saves significant time during surgery, as the cannula needs not be introduced before the knife, since the cannula and the knife are combined. The combined cannula/knife system of the present invention also protects the soft tissue and organs adjacent the cut, as the knife is retracted up into the protective sleeve once it has been introduced into the patient.

The present invention also provides a method of penetrating tissue by inter alia: (i) providing a combined cannulated dilation sleeve integrated with a knife in the proximity of tissue to be cut or punctured; and (ii) deploying the knife from a lumen of the sleeve to cut or puncture tissue.

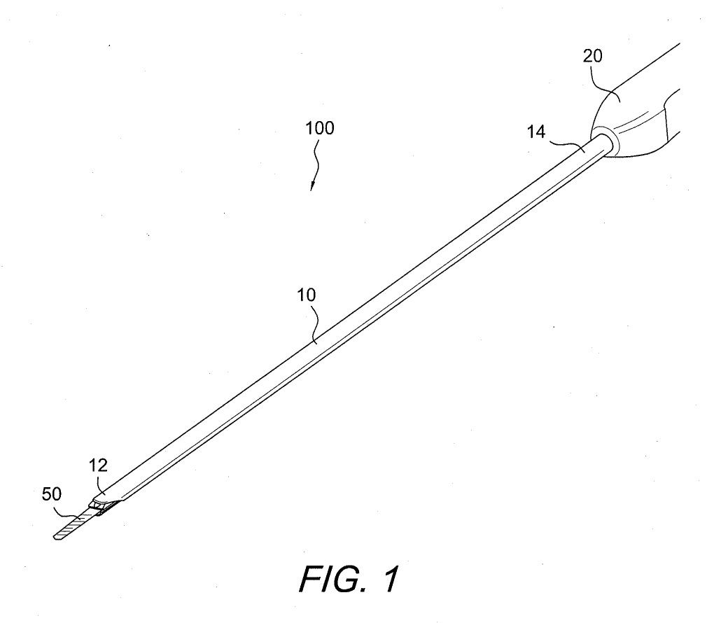

Referring now to the drawings, where like elements are designated by like reference numerals, FIGS. 1-10 illustrate a combined cannulated sleeve/knife 100 of the present invention formed of a tubular member or cannulated sleeve 10 (cannulated dilation sleeve 10) which receives a knife assembly 5 with a knife 50 (blade or scalpel 50) configured to retract within a lumen of the tubular member or sleeve 10. Instrument 100 also includes a handle 20 (FIG. 1) securely attached to the tubular member or cannulated sleeve 10 and provided with an operating mechanism (such as a trigger, spring or friction slide, for example) configured to actuate (advance or retract) the knife assembly 5 or, more preferably, to advance or retract the cannulated sleeve relative to the knife assembly. In this manner, the blade or scalpel 50 may be disposed in at least two positions relative to the tubular member of sleeve 10: a retracted or non-working position (in which the knife assembly 5 with blade 50 is partially or totally retracted within the lumen of the tubular member or sleeve 10) and an extended or working position (in which the knife assembly 5 and blade 50 partially or totally extends a distance beyond the most distal end of the tubular member or sleeve 10).

As shown in FIG. 1, the tubular member or cannulated sleeve 10 has a distal end 12 and a proximal end 14. Knife or blade 50, mounted on knife assembly 5, is configured to exit a lumen at a most distal end 12 of the sleeve 10 and to extend at least a distance beyond the most distal end 12 of the sleeve (as shown in FIG. 4, for example). The tubular member or cannulated sleeve 10 may be formed from a medically acceptable material such as stainless steel, and may have a round, cylindrical or ellipsoidal cross-section, among others. Preferably, the cannulated sleeve is tubular and has a round cross-section.

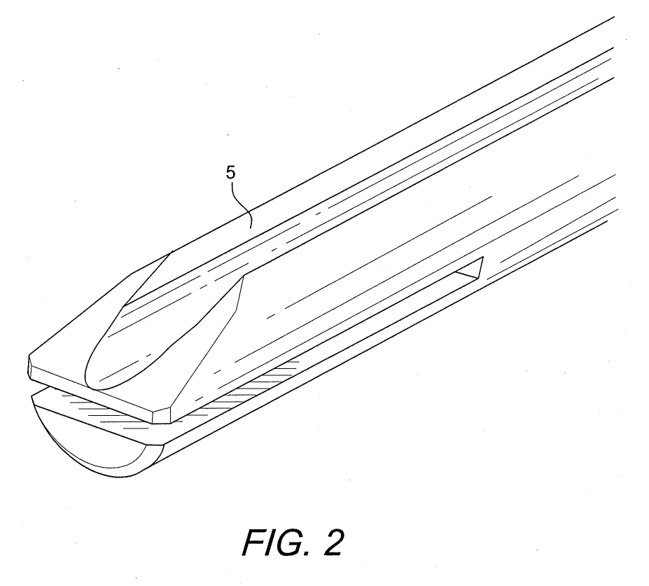

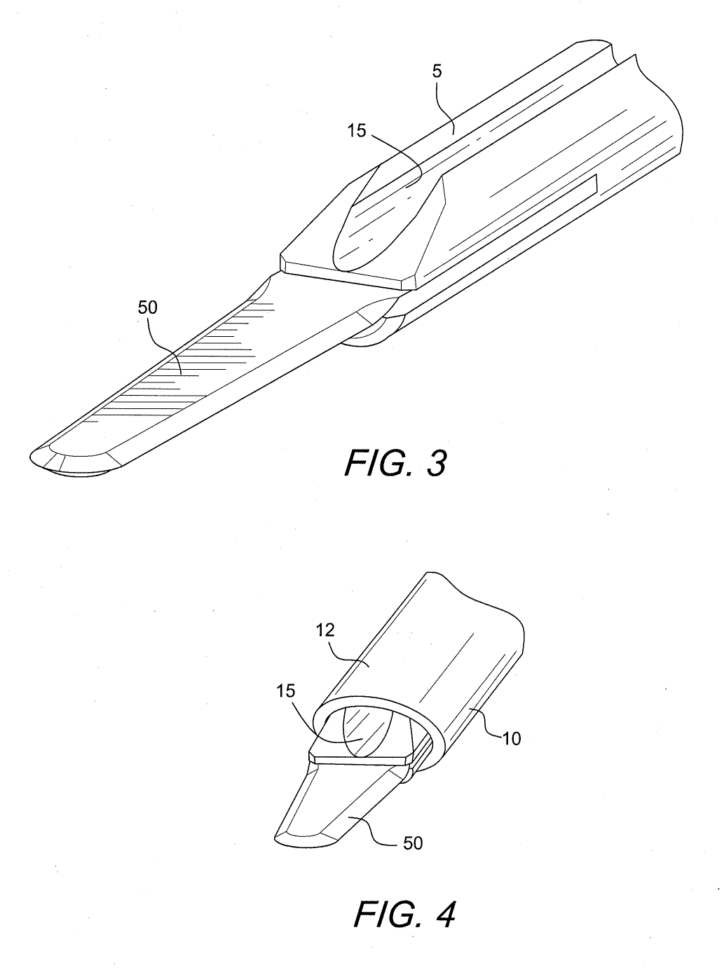

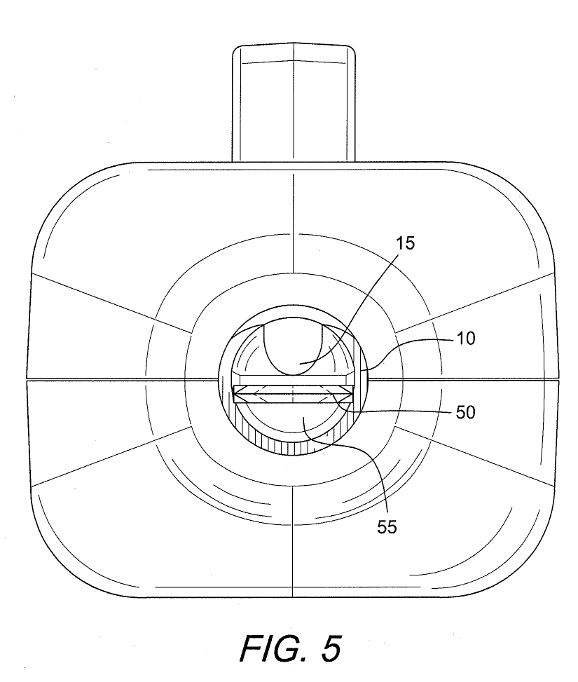

FIG. 2 illustrates knife assembly 5 without a knife or blade 50 installed (pre-assembly), while FIG. 3 shows knife assembly 5 fully assembled with knife 50. FIG. 4 illustrate knife or blade 50 deployed from the cannulated sleeve 10 (in a working, cutting or extended position). FIG. 5 illustrates a view taken from the most distal end 12 of the sleeve 10. When the knife or blade 50 is in the non-working or non-cutting (inoperative) position, the blade is at least partially (preferably totally) shielded within a lumen of the cannulated sleeve 10. When the knife or blade 50 is in the working or cutting (operative) position, the blade is projected from the lumen of the cannulated sleeve 10. In this manner, the blade is protected when the knife is not being used or is being retrieved prior to the actual cutting. The user may then choose between the retracted blade position (shown in FIG. 2) and the extended, deployed position (shown in FIGS. 3 and 4). In the extended (working or cutting position), blade 50 is about parallel to (aligned with) a longitudinal axis 10a (shown schematically in FIG. 10) of the cannulated sleeve 10.

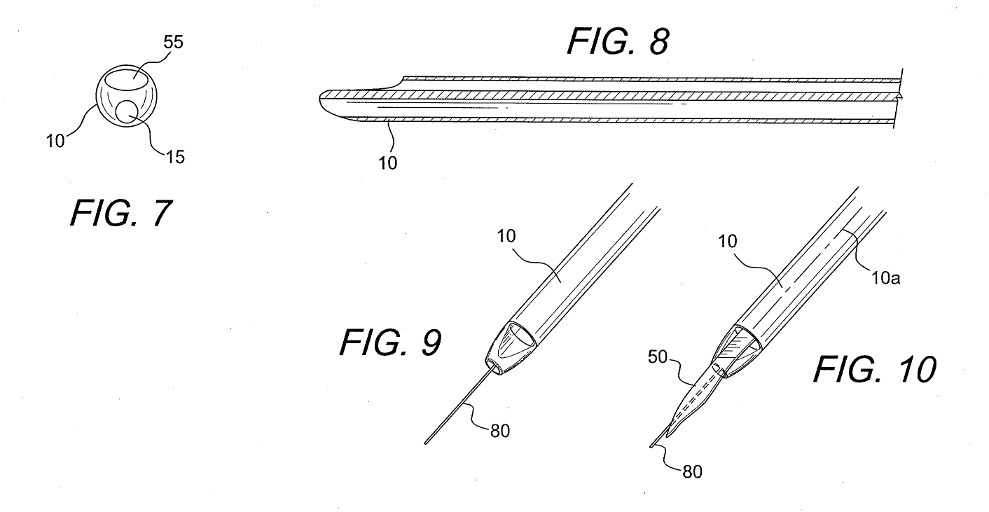

As shown in FIGS. 5 and 7, sleeve 10 is configured as a multiple-lumen tube stock, for example, as a double-lumen tube 10 comprising two lumens or two cannulations, i.e., a first lumen or cannulation 55 adjacent a second lumen or cannulation 15 (which can be formed in the knife assembly 5, as shown). In an exemplary embodiment, one of the first and second cannulations 55, 15 houses a first surgical instrument (for example, a cutting blade or scalpel 50) and the other of the first and second cannulations 55, 15 allows passage of a second surgical instrument (for example, a guide wire 80) or may act as an aspiration/irrigation port (so that any fluid and/or loose tissue resulting from the cutting procedure can be aspirated through one of the lumens of the multiple-lumen sleeve 10).

In an exemplary embodiment, the first lumen or cannulation 55 is a blade lumen or blade cannulation 55 that houses knife assembly 5 with blade 50, and the second lumen or cannulation 15 is a guide wire lumen or guide wire cannulation 15 that houses guide wire 80. Knife assembly 5 with blade 50 is configured to slide along (to retract and exit) within the blade cannulation 55. The guide wire cannulation 15 slides over the guide wire 80.

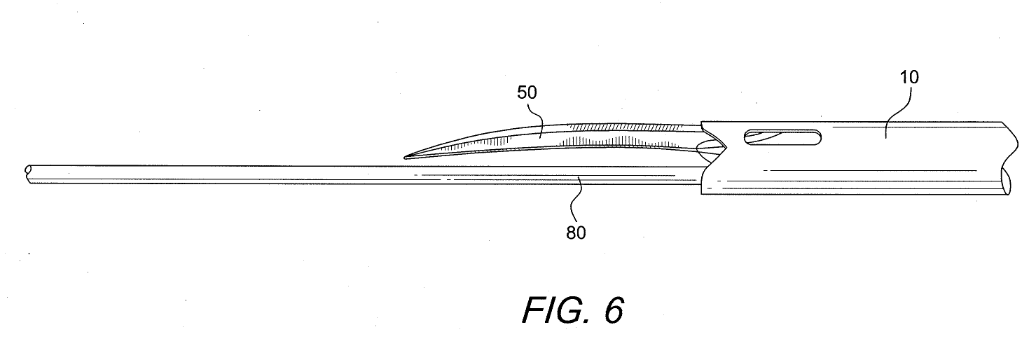

The first lumen or cannulation 55 may have a cross-section similar to, or different from, that of the second lumen or cannulation 15. In an exemplary embodiment only, and as shown in FIGS. 5 and 7, blade cannulation 55 may be oval or elliptical and is located adjacent the guide wire cannulation 15. Guide wire cannulation 15 may be preferably round and allows a flexible guide wire (or another surgical instrument) to be introduced therethrough. FIGS. 6 and 10 illustrate flexible guide wire 80 adjacent blade or knife 50 (shown in the deployed configuration), with the guide wire 80 extending about parallel to blade or knife 50 and to the longitudinal axis 12a.

In an exemplary embodiment only, the combined cannulated sleeve/knife 100 of the present invention is an integrated guide wire/blade device. The blade can extend through one of the lumens of the device and, in use, the blade can be selectively movable between a retracted position where the blade is in a constrained configuration within the lumen and an extended position in which a portion of the blade extends a distance beyond a most distal end of the lumen. The portion of blade that extends the distance beyond the distal end of the lumen cuts and/or penetrates tissue or structures adjacent to the tissue.

The integrated cannula/knife instrument 100 described above may be employed in various surgical medical procedures such as conventional open surgeries or in other, less invasive, techniques that use cannulas or various port access devices. The present invention has applications in surgical procedures where the target tissue is cut or punctured, and may be employed in cutting various body parts such as the knee, shoulder, hip, ankle, elbow, hand or foot. For example, the integrated cannula/knife instrument 100 of the present invention may be employed in arthroscopic surgery of a knee joint or hip structure.

A method of cutting/penetrating tissue with the cannulated arthroscopic knife of the present invention comprises inter alia the steps of: (i) providing a combined cannulated dilation sleeve 10 integrated with a knife 50, in the proximity of tissue to be cut or punctured; and (ii) deploying the knife 50 from a lumen 55 of the sleeve 10, to cut or puncture tissue.

In an exemplary and illustrative embodiment only, a method of portal placement and capsular resection during hip arthroscopy according to the present invention comprises the steps of: (i) introducing a spinal needle; (ii) introducing a flexible guide wire 80 through the needle; (iii) removing the needle, leaving the guide wire 80 in place; (iv) sliding a cannulated retractable arthroscopic knife 100 over the guide wire 80 and into the joint, with the knife blade 50 retracted back into sleeve 10; (v) removing the guide wire 80; (vi) deploying the knife 50 to cut the capsule; (vii) retracting the knife blade 50 into the housing 55, and reintroducing the guide wire 80 through the knife cannulation 15; and (viii) removing the retractable knife 100 from the joint, leaving the guide wire 80 in place.

Although the present invention has been described in relation to particular embodiments thereof, many other variations and modifications and other uses will become apparent to those skilled in the art. Therefore, the present invention is to be limited not by the specific disclosure herein, but only by the appended claims.

1. A surgical cutting instrument, comprising:
a double-lumen tubular member having a distal end, a proximal end, and a longitudinal axis; and
wherein the double-lumen tubular member comprises a first lumen adjacent and parallel to a second lumen, wherein one of the first and second lumens houses a retractable cutting blade which is movable between an inoperative or retracted position within the tubular member and an operative or extended position wherein the cutting blade extends a distance beyond a most distal end of the double-lumen tubular member.

2. The surgical cutting instrument of claim 1, wherein the double-lumen tubular member is a dilation sleeve.

3. The surgical cutting instrument of claim 1, wherein the first lumen has a different cross-section than the second lumen.

4. The surgical cutting instrument of claim 1, wherein the other of the first and second lumens is an aspiration or irrigation channel.

5. The surgical cutting instrument of claim 1, wherein the other of the first and second lumens houses a surgical instrument different from the cutting blade.

6. The surgical cutting instrument of claim 4, wherein the surgical instrument is a guide wire.

7. The surgical cutting instrument of claim 1, wherein first lumen has a circular cross-section and the second lumen has an oval cross-section.

8. The surgical cutting instrument of claim 1, wherein the cutting blade is employed in ACL or hip surgery.

9. An integrated guide wire/knife device, comprising:
an elongate tubular member having a distal end, a proximal end, a longitudinal axis and two distal openings located at the distal end;
wherein the elongate tubular member comprises a first lumen adjacent a second lumen, the first and second lumens extending within the elongate tubular member and about parallel to each other and to the longitudinal axis, each of the first and second lumens terminating in one of the two distal openings, wherein one of the first and second lumens houses a blade which extends between an inoperative or retracted position and an operative or extended position, and the other the of the first and second lumens houses a guide wire.

10. The integrated guide wire/knife device of claim 9, wherein the elongate tubular member is a dilation sleeve.

11. The integrated guide wire/knife device of claim 9, wherein the elongate tubular member is formed of stainless steel.

12. The integrated guide wire/knife device of claim 9, wherein the first lumen has a cross-section different from the second lumen.

13. A method of conducting surgery, comprising the steps of:
providing an integrated device comprising a cannulated dilation sleeve integrated with a blade, in the proximity of tissue to be cut or punctured; and
deploying the blade from a lumen of the sleeve to cut or puncture tissue.

14. The method of claim 13, wherein the tissue is capsular tissue of the hip joint.

15. The method of claim 13, wherein the cannulated dilation sleeve comprises a first lumen that houses the blade and an adjacent second lumen that houses another surgical instrument.

16. The method of claim 15, wherein the another surgical instrument is a guide wire.

17. A method of capsular resection during hip arthroscopy comprising the steps of:
introducing a spinal needle into a hip joint;
introducing a flexible guide wire through the needle;
removing the needle, leaving the guide wire in place;
providing a retractable knife device in the proximity of the guide wire, the retractable knife device comprising a double-lumen sleeve and a retractable blade housed within a lumen of the double-lumen sleeve;
sliding the retractable knife device over the guide wire and into the hip joint, with the blade retracted back into the double-lumen sleeve;
removing the guide wire;
deploying the blade so that the blade exits the lumen of the double-lumen sleeve and extends beyond a most distal end of the double-lumen sleeve; and
cutting capsular tissue with the blade.

18. The method of claim 17, further comprising the steps of:
retracting the blade into the lumen, and reintroducing the guide wire through another lumen of the sleeve; and
removing the retractable knife device from the joint, leaving the guide wire in place.

19. The method of claim 17, wherein the double-lumen sleeve is a dilation sleeve.

20. The method of claim 17, wherein the double-lumen sleeve has a first lumen with a round cross-section and a second lumen with an oval cross-section.
